(19) United States
(12) Patent Application Publication
(10) Pub. No.: US 2024/0341366 A1
(43) Pub. Date: Oct. 17, 2024

(54) DELIVERY DEVICE AND SYSTEM

(71) Applicant: Nicoventures Trading Limited, London (GB)

(72) Inventors: Harry GREEN, London (GB); Anna AZZOPARDI, London (GB)

(21) Appl. No.: 18/682,694

(22) PCT Filed: Aug. 10, 2022
(86) PCT No.: PCT/GB2022/052079

§ 371 Date: Feb. 9, 2024

(30) Foreign Application Priority Data
Aug. 12, 2021 (GB) 2111602.5

(51) Int. Cl. A24F 40/53; A24F 40/51

(52) U.S. Cl. CPC A24F 40/53; A24F 40/51

(57) ABSTRACT

There is provided a delivery system for a metabolic modulator comprising: a sensor component configured to determine a metabolic state of a user; a delivery component configured to deliver a metabolic modulator; and a controller configured to receive information relating to the metabolic state of the user and to control delivery of the metabolic modulator in response thereto.

### FIELD

The present disclosure relates to a delivery device for delivering a metabolic modulator and a delivery system comprising the delivery device.

### BACKGROUND

Human energy balance is crucial in understanding weight gain and weight loss, and successful weight loss results from matching energy intake to energy output. In general, energy expenditure (EE) is a measure of the amount of energy required for various biological functions. Resting EE (REE) is the energy needed to support minimal cellular processes and comprises approximately two-thirds of total daily EE (TDEE). The most common method of measuring EE is through indirect calorimetry. Oxygen consumption and carbon dioxide expiration are measured in order to determine EE by the organism. This is directly related to the energy needs as a function of the cellular processes.

Understanding and modulating EE is a factor in weight gain and weight loss and it would be desirable to provide advances in this area.

### SUMMARY

In one aspect of the present disclosure there is provided a delivery system for a metabolic modulator comprising: a sensor component configured to determine a metabolic state of a user; a delivery component configured to deliver a metabolic modulator; and a controller configured to receive information relating to the metabolic state of the user and to control delivery of the metabolic modulator in response thereto

In a further aspect, there is provided a controller for a delivery system for a metabolic modulator, the controller configured to: receive information relating to the metabolic state of the user from a sensor component configured to determine a metabolic state of a user; and control delivery of the metabolic modulator via in response thereto.

In a further aspect, there is provided a delivery component for a delivery system for a metabolic modulator, the delivery component configured to deliver a metabolic modulator in response to information relating to the metabolic state of the user

In one aspect of the present disclosure there is provided a method of operating a delivery system for a metabolic modulator, the delivery system comprising a sensor component configured to determine a metabolic state of a user, a delivery component configured to deliver a metabolic modulator and a controller configured to receive information relating to the metabolic state of the user and to control delivery of the metabolic modulator in response thereto, the method comprising: determining a metabolic state of a user; and receiving information relating to the metabolic state of the user; and controlling delivery of the metabolic modulator in response thereto.

In a further aspect of the present disclosure there is a means for delivering a metabolic modulator comprising: sensor means configured to determine a metabolic state of a user; delivery means configured to deliver a metabolic modulator; and control means configured to receive information relating to the metabolic state of the user and to control delivery of the metabolic modulator in response thereto.

These and other aspects as apparent from the following description form part of the present disclosure. It is expressly noted that a description of one aspect may be combined with one or more other aspects, and the description is not to be viewed as being a set of discrete paragraphs which cannot be combined with one another.

### BRIEF DESCRIPTION OF THE DRAWINGS

FIG. 1 is a schematic representation of an aerosol provision device according to the present disclosure.

FIG. 2 is an abstract graphical representation of the metabolic rate of three example users with respect to time.

FIG. 3 is a schematic diagram of an exemplary delivery system in accordance with the present invention.

FIG. 4 is a further schematic diagram of an exemplary delivery system in accordance with the present invention.

FIG. 5 is a still further schematic diagram of an exemplary delivery system in accordance with the present invention.

FIG. 6 is a still yet further schematic diagram of an exemplary delivery system in accordance with the present invention.

FIG. 7 schematically represents a method of controlling an aspect of the delivery system in accordance with certain embodiments of the disclosure.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

In accordance with example embodiments of the present disclosure, a delivery system is provided having a sensor configured to determine the metabolic state of a user; and a delivery device comprising a controller and at least one metabolic modulator, wherein the controller is configured to receive information relating to the metabolic state of the user and to control delivery of the at least one metabolic modulator to the user in response thereto. A delivery system configured in this way is operable to respond to provide a level of control of the metabolic state of a user.

By metabolic state of the user, it is meant an indication or measure of the energy expenditure (EE) of a user. The metabolic state may be referred to as a metabolic rate, indicating the amount of energy used (i.e. expended) per unit of time (e.g. kcal per hour). A user having a high metabolic rate expends more energy per unit time (i.e. they have a higher energy expenditure) than a user having a low metabolic rate. As such, the metabolic state of the user determines the amount of energy used by the user. Therefore, providing a level of control of the metabolic state advantageously allows the promotion or maintenance of the energy expenditure of a user within a target range.

In some examples, at least one metabolic modulator can be delivered or otherwise provided to a user to induce a metabolic state which is higher than the metabolic state would be without the metabolic modulator, thereby increasing the energy usage by the user. An increased energy usage may aid weight management (e.g. by enhancing weight loss, or by aiding stabilisation of a user's weight).

In some examples, at least one metabolic modulator can be delivered or otherwise provided to a user to induce a metabolic state which is lower than the metabolic state would be without the metabolic modulator, thereby decreasing the energy usage by the user. A decreased energy usage may aid weight management (e.g. by enhancing weight gain, or by aiding stabilisation of a user's weight)

In some further examples, the delivery or provision of at least one metabolic modulator can be restricted or limited, to produce a reduced change in the metabolic rate of a user. Limiting change to a user's metabolic rate can be used to aid targeting the user's metabolic rate which can be useful for weight management (e.g. by preventing a higher or lower than desired metabolic rate).

### Metabolic Modulators

The metabolic modulator as used herein may be a physiologically active material, which is a material intended to achieve or enhance a physiological response on the metabolism of a user. The metabolic modulator may be naturally occurring or synthetically obtained. The metabolic modulator may comprise for example nicotine, caffeine, taurine, theine, vitamins such as B6 or B12 or C, melatonin, cannabinoids, or constituents, derivatives, or combinations thereof. The metabolic modulator may comprise one or more constituents, derivatives or extracts of tobacco, cannabis or another botanical.

In some examples, a metabolic modulator may comprise one selected from the group comprising Nicotine, Caffeine, catecholamine, Cannabinoids, L-theanine, and Terpenes. It will be appreciated that other compounds having an effect on metabolic state could be used.

In some examples, the metabolic modulator may be a metabolic stimulant such as Nicotine, caffeine, and catecholamine. By metabolic stimulant it is meant a compound which increases (i.e. stimulates) the metabolic rate of the user, and hence increases energy usage.

As stated above, in some examples catecholamines (e.g. norepinephrine and epinephrine) are used as a metabolic stimulant. Catecholamines stimulate beta-1 and beta-2 adrenergic receptors eliciting a fight-or-flight response. The paraventricular nucleus of hypothalamus contains adrenoreceptors that when stimulated by norepinephrine, decrease food intake. In addition to inducing hypophagia, these catecholamines are part of a collective stress response and stimulation of the agonistic receptors centrally and peripherally mobilizes the energy stores (adipose tissue), increasing the caloric demand of the body and overall oxygen consumption.

As stated above, in some examples nicotine is used as a metabolic stimulant. Nicotine has demonstrated an increase of energy expenditure and there is general agreement that this occurs primarily as a consequence of catecholamine stimulation. Nicotine has been shown to stimulate the release of norepinephrine both inside and outside of the hypothalamus. The utilization of energy stores as a consequence of catecholamine stimulation from nicotine further explains how nicotine treatment demonstrates decreased adiposity in animals. In some embodiments, nicotine is a derivative or extract of tobacco.

As stated above, in some examples, caffeine is used as a metabolic modulator due to its stimulating effect on the metabolic rate of a user.

In some examples two or more metabolic stimulants may be used in combination to provide an enhanced effect on the metabolic rate. For example, in some examples, caffeine is used in combination with nicotine. The combination of caffeine and nicotine significantly enhances the thermogenic response on energy expenditure. Glucose and fat oxidation rates were similar regardless of the dose indicating that this response is not a function of changes in substrate oxidation [144].

In some examples, the metabolic modulator may be a metabolic suppressant such as Cannabinoids, L-theanine, and Terpenes. By metabolic suppressant it is meant a compound which decreases (i.e. supresses) the metabolic rate of the user, and hence decreases energy usage.

As noted herein, the metabolic modulator may comprise one or more constituents, derivatives or extracts of cannabis, such as one or more cannabinoids or terpenes. For example, as stated above, cannabinoids can be used as a metabolic modulator due to the inhibiting effect on the metabolic rate of the user. For example, as also stated above, terpenes can be used as a metabolic modulator due to the inhibiting effect on the metabolic rate of the user.

As stated above, in some examples, L-theanine can be used as a metabolic modulator due to the inhibiting effect on the metabolic rate of the user.

As noted herein, the metabolic modulator may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Memtha longifolia, Mentha suaveolens variegata, Mentha pulegium, Mentha spicata c.v. and Mentha suaveolens

In some embodiments, the metabolic modulator comprises or derived from one or more botanicals or constituents, derivatives or extracts thereof and the botanical is selected from eucalyptus, star anise, cocoa and hemp.

In some embodiments, the metabolic modulator comprises or derived from one or more botanicals or constituents, derivatives or extracts thereof and the botanical is selected from rooibos and fennel.

In some examples two or more metabolic stimulants may be used in combination to provide an enhanced effect on the metabolic rate.

In some examples a metabolic stimulant may be used in combination with a metabolic suppressant to produce a moderated effect on the metabolism. For example, a combination can be provided such that the effect of a metabolic stimulant is dampened due to the simultaneously intake due to a metabolic suppressant. Alternatively, in some examples, a combination of compounds can be provided to a user such that the effect of a metabolic suppressant is dampened due to the simultaneously intake due to a metabolic stimulant. It will further be appreciated that various combinations of 3 or more compounds (either stimulants or depressants) could be provided to allow a more tailored effect on metabolic rate.

Physical activity and Metabolic Rate

As well as being dependent on the effect of metabolic modulators, the metabolic rate is influenced by physical activity. Hence physical activity can be used to indirectly infer a metabolic rate in comparison to a baseline (e.g. by determining whether a user is at rest or active, and estimating a metabolic rate depending on how “active” the user is).

The combination of physical activity (i.e. how active a user is) and the delivery of a metabolic modulator can have an improved effect over the delivery of a metabolic modulator to an inactive user (i.e. a user at rest). Without being bound by theory, the effect of a metabolic modulator can be enhanced by the user being in an active state rather than a rest state.

For example, the pharmacokinetics of nicotine result in enhanced metabolic effects of physical activity. This may be due to physical activity slowing nicotine metabolism, prolonging the presence of nicotine in the blood, thereby increasing the catecholaminergic effect.

In view of the above, in some examples, the physical activity (e.g. active state) of the user can be considered as an additional factor when determining how to control a user's metabolic rate (e.g. by determining an appropriate amount of at least one metabolic modulator to delivery or otherwise provide to a user).

### Delivery Device

The term “delivery device” is intended to encompass devices or systems that deliver at least one substance to a user, and includes:

non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and aerosol-free delivery devices that deliver the at least one substance to a user orally, nasally, transdermally or in another way without forming an aerosol, including but not limited to patches, articles comprising inhalable powders, and oral products, wherein the at least one substance may or may not comprise nicotine.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery device is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

In some embodiments, the substance to be delivered comprises the metabolic modulator.

In some embodiments, the substance to be delivered comprises a flavour.

As used herein, the terms “flavour” and “flavourant” refer to materials which, where local regulations permit, may be used to create a desired taste, aroma or other somatosensorial sensation in a product for adult consumers. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis.

In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

Aerosol-generating material is a material that is capable of generating aerosol, for example when heated, irradiated or energized in any other way. Aerosol-generating material may, for example, be in the form of a solid, liquid or gel which may or may not contain an active substance and/or flavourants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it.

In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavours, one or more aerosol-former materials, and optionally one or more other functional material.

The aerosol-former material may comprise one or more constituents capable of forming an aerosol. In some embodiments, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, colouring agents, preservatives, binders, fillers, stabilizers, and/or antioxidants.

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The device that is configured to generate the varying magnetic field is referred to as a magnetic field generator, herein.

An aerosol-modifying agent is a substance, typically located downstream of the aerosol generation area, that is configured to modify the aerosol generated, for example by changing the taste, flavour, acidity or another characteristic of the aerosol. The aerosol-modifying agent may be provided in an aerosol-modifying agent release component that is operable to selectively release the aerosol-modifying agent

The aerosol-modifying agent may, for example, be an additive or a sorbent. The aerosol-modifying agent may, for example, comprise one or more of a flavourant, a colourant, water, and a carbon adsorbent. The aerosol-modifying agent may, for example, be a solid, a liquid, or a gel. The aerosol-modifying agent may be in powder, thread or granule form. The aerosol-modifying agent may be free from filtration material.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

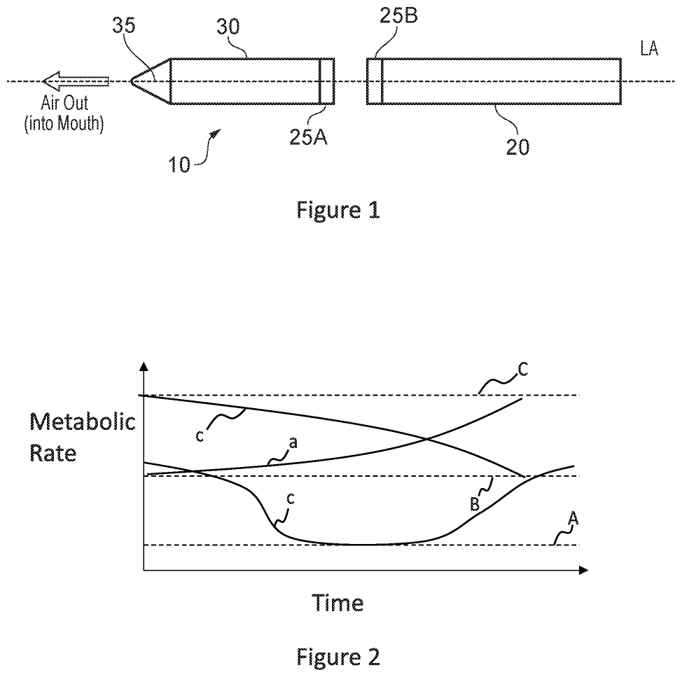

FIG. 1 is a schematic diagram of an exemplary aerosol/vapour provision system (not to scale) in accordance with the present invention. The exemplary e-cigarette 10 has a generally cylindrical shape, extending along a longitudinal axis indicated by dashed line LA, and comprising two main components, namely a body 20 (aerosol provision device) and a cartomiser 30. The cartomiser includes an internal chamber containing a reservoir of a source liquid comprising a liquid formulation from which an aerosol is to be generated, a heating element (which is an example of an aerosol generator), and a liquid transport element (in this example a wicking element) for transporting a source of (one or more) metabolic modulator(s) in the form of a liquid to the vicinity of the heating element. The heating element, a portion of the liquid transport element and a volume surrounding the heating element and the portion of the liquid transport element may be referred to as the aerosol generation region (i.e., the region in which an aerosol is generated).

The cartomiser 30 further includes a mouthpiece 35 having an opening through which a user may inhale the aerosol from the heating element. The source liquid may be of a conventional kind used in e-cigarettes, for example comprising 0 to 5% nicotine dissolved in a solvent comprising glycerol, water, and/or propylene glycol. The source liquid may also comprise flavourings. The reservoir for the source liquid may comprise a porous matrix or any other structure within a housing for retaining the source liquid until such time that it is required to be delivered to the aerosol generator/vaporiser. In some examples the reservoir may comprise a housing defining a chamber containing free liquid (i.e. there may not be a porous matrix).

As discussed further below, the body 20 includes a re-chargeable cell or battery to provide power for the e-cigarette 10 and a circuit board including control circuitry for generally controlling the e-cigarette. In active use, i.e. when the heating element receives power from the battery, as controlled by the control circuitry, the heating element vaporises source liquid in the vicinity of the heating element to generate an aerosol. The aerosol is inhaled by a user through the opening in the mouthpiece. During user inhalation the aerosol is carried from the aerosol generation region to the mouthpiece opening along an air channel that connects between them.

In the exemplary system of FIG. 1, the body 20 and cartomiser 30 are detachable from one another by separating in a direction parallel to the longitudinal axis LA, as shown in FIG. 1, but are joined together when the device 10 is in use by a connection, indicated schematically in FIG. 1 as 25A and 25B, to provide mechanical and/or electrical connectivity between the body 20 and the cartomiser 30. The electrical connector on the body 20 that is used to connect to the cartomiser may also serve as a socket for connecting a charging device (not shown) when the body is detached from the cartomiser 30. The other end of the charging device can be plugged into an external power supply, for example a USB socket, to charge or to re-charge the cell/battery in the body 20 of the e-cigarette. In other implementations, a cable may be provided for direct connection between the electrical connector on the body and the external power supply and/or the device may be provided with a separate charging port, for example a port conforming to one of the USB formats.

The e-cigarette 10 is provided with one or more holes (not shown in FIG. 1) for use as an air inlet. These holes connect to an air passage (airflow path) running through the e-cigarette 10 to the mouthpiece 35. Typically the air path through such devices is relatively convoluted in that it has to pass various components and/or take multiple turns following entry into the e-cigarette. The air passage includes a region around the aerosol generation region and a section comprising an air channel connecting from the aerosol generation region to the opening in the mouthpiece.

When a user inhales through the mouthpiece 35, air is drawn into this air passage through the one or more air inlet holes, which are suitably located on the outside of the e-cigarette. This airflow (or the associated change in pressure) may be detected by an airflow sensor (not shown), in this case a pressure sensor, for detecting airflow in electronic cigarette 10 and outputting corresponding airflow detection signals to the control circuitry. The airflow sensor may operate in accordance with conventional techniques in terms of how it is arranged within the electronic cigarette to generate airflow detection signals indicating when there is a flow of air through the electronic cigarette (e.g. when a user inhales or blows on the mouthpiece).

When a user inhales (sucks/puffs) on the mouthpiece in use, the airflow passes through the air passage (airflow path) through the electronic cigarette and combines/mixes with the vapour in the region around the aerosol generation region to generate the aerosol. The resulting combination of airflow and aerosol continues along the airflow path connecting from the aerosol generation region to the mouthpiece for inhalation by a user. The cartomiser 30 may be detached from the body 20 and disposed of when the supply of source liquid is exhausted (and replaced with another cartomiser if so desired). Alternatively, the cartomiser may be refillable.

### Sensors

The term “sensor” is intended to encompass devices that measure at least one physical characteristic (e.g. parameter or variable), and in particular a characteristic related to, or indicative of, a user's metabolism when in use.

An example characteristic is heart rate which can be measured by, for example, a heart rate sensor (e.g. an vibration or optical sensor for detecting pulses). Heart rate can be used to identify a state of activity of the user (e.g. “active” or “at rest”) which can then be used to infer a metabolic state of the user.

An example characteristic is body temperature which can be measured by, for example, a thermometer or temperature sensor (e.g. a thermistor, or an optical sensor, such as a infrared optical sensor). Body temperature can be indicative of the energy expenditure of the body.

An example characteristic is physical motion which can be measured by, for example, a motion sensor (e.g. an accelerometer or gyroscope). Physical activity can be used to identify a state of activity of the user (e.g. “active” or “at rest”) which can then be used to infer a metabolic state of the user.

An example characteristic is blood pressure which can be measured by, for example, a blood pressure sensor (e.g. a vibration or pressure sensor). Blood pressure can be used to identify a state of activity of the user (e.g. “active” or “at rest”) which can then be used to infer a metabolic state of the user.

An example characteristic is oxygen as measured by a chemical sensor. Users consume oxygen as part of metabolic processes and therefore a higher level of oxygen consumption indicates a higher metabolic rate.

An example characteristic is carbon dioxide as measured by a chemical sensor. Users produce carbon dioxide as they consume oxygen as part of metabolic processes and therefore a higher level of carbon dioxide production indicates a higher metabolic rate.

An example characteristic is respiration rate which can be measured by, for example, a motion (e.g. an accelerometer), acoustic (e.g. vibration sensor), or pressure sensor. Users consume oxygen and produce carbon dioxide as part of metabolic processes and therefore a higher respiration rate can be indicative of an increased need for oxygen which is itself indicative of a higher metabolic rate. A measurement of respiration rate could further be improved by estimating or determining the volume or air inhaled and exhaled.

An example characteristic is rapid eye movement during sleep (i.e. REM sleep), which can be measured by electroencephalography, or can be inferred from a motion sensor.

In examples a sensor, which may be a sensor configured to measure one or more of the above example characteristics, is configured to output the information directly or indirectly relating to the metabolic state of the user. As such, the sensor produces information or data from which the metabolic state of a user can be calculated, inferred or estimated. In some examples, the sensor, or a processing component in communication with the sensor and receiving readings from the sensor, can perform calculations on the measured data to determine or estimate the metabolic state before outputting the calculated information relating to the metabolic state of the user.

In some examples, the sensor can measure multiple physical characteristics and/or can work in conjunction with one or more other sensors for measuring physical characteristics. For example, a sensor can be configured to measure both heart rate and user activity. A higher heart rate in the absence of increased user activity can indicate a higher REE. In some examples, a first sensor can be placed in a preferential position on a user for measuring heart rate and a second sensor can be placed in a preferential position on a user for measuring movement. The first and second sensor can work in conjunction with each other to allow the determination of a metabolic state of the user. In some examples, a first sensor can be placed in a first position on a user and a second sensor can be placed in a second position on a user, and both sensors can be measure the same characteristic. This can improve the reliability of the measurement. It will be appreciated that in general combining measurements of multiple characteristics can be used to improve the determination of the metabolic state of the user.

### Control Mechanisms

In accordance with example embodiments of the present disclosure, the delivery system comprises a controller configured to receive information or data relating to the metabolic state of the user from at least one sensor. The controller is configured to control delivery of at least one metabolic modulator to the user in response to, or based on, the information relating to the metabolic state of the user. A delivery system having a controller configured in this way is operable to respond to a information relating to a metabolic state of the user to provide a level of control of the metabolic state of a user.

In some examples, the controller can compare the information received from the sensor, relating to the metabolic state of the user, to baseline information relating to a resting metabolic state of the user. In some of these examples, the baseline information can be recorded during a calibration process or initialisation process by the at least one sensor and provided to the controller. In others of these examples, the user can provide or otherwise input baseline information to the controller; e.g. via a user interface. The controller can compare the information with the baseline information to determine or estimate the metabolic state of the user. For example, a heart rate below, slightly above, or equal to the baseline resting heartrate can indicate a low metabolic state. Similarly a heart rate significantly above the resting heartrate can indicate a high metabolic state.

In some examples, the controller can periodically or continuously compare information received from the sensor (i.e. sensor readings or measurements), relating to the metabolic state of the user, to previous information relating to the metabolic state of the user at a previous time. The comparison can be used to determine a current metabolic state and/or a rate of change in metabolic state. Furthermore, to provide an improved determination the controller can base a rate of change on multiple sets of information relating to the metabolic state of the user at multiple previous times. In some examples, at least a portion of the previous information relating to metabolic state is measured using the same sensor as the sensor providing the controller with information periodically or continuously.

In some examples, at least a portion of the previous information relating to metabolic state is measured using a different sensor to the sensor providing the controller with information periodically or continuously. For example, the previous information may be measured by a first sensor which is more accurate and/or makes a more direct measurement of the metabolic state to allow the formulation of a reliable baseline for a user. Such a first sensor may be bulkier, power hungry, and/or less convenient to carry by a user. A second sensor, measuring a characteristic from which metabolic rate can be indirectly estimated or inferred, is then used to estimate or infer a metabolic rate based on the baseline. Such a second sensor may be smaller, power efficient and/or more convenient to carry by a user). In some examples, the second sensor can measure concurrently with the first sensor to enable a more accurate relationship to be derived between the measurements of the metabolic rate using the first sensor, and the measurements of the metabolic rate using the second sensor.

In some examples, the time between the measurement of the current information and the measurement of the previous information can be between 1 minute and 60 minutes before the current information was supplied, between 5 and 40 minutes before the current information was supplied, between 10 and 30 minutes before the current information was supplied. It will be appreciated that in some examples, far shorter time periods may be use. For example, some sensors can measure a parameter related at a frequency of around several thousand Hertz. In some examples, the sensors may communicate all of the measured data or information to the controller which can analyse the data to identify trends. In other examples, the sensors perform analysis themselves and communicate the analysed data or information to the controller.

In some examples, the controller is configured to determine whether a (dose of) one or more metabolic modulators should be delivered to the user and/or a time at which a one or more metabolic modulators should be delivered to the user based on the received information relating to the metabolic state of the user. In some examples, the controller may automatically deliver the one or more metabolic modulators to the user, immediately or at the determined time, based on determining that the one or more metabolic modulators should be delivered to the user.

In some examples, the controller can receive information relating to the metabolic state of the user and can alter the deliverance of one or metabolic modulators to the user in response to receiving the information. In some examples, the controller is configured to use the received information relating to perform a comparison. In some examples, the received information is indicative of a user's metabolic state and can be compared to one or more comparison values to determine how to control the delivery of at least one metabolic modulator upon receipt. In some examples, the received information corresponds to, or is otherwise associated with, a metabolic rate of the user such that a metabolic rate of the user is established upon receiving the information. In other examples, the controller is configured to establish a metabolic rate of the user by performing further analysis or processing. In some examples, the received information is processed or analysed by the controller to take account of systematic corrections dependent on the sensor or ambient conditions. In some examples, the received information is processed or analysed to convert, or generate from, the received information a new data format that is more readily comparable.

In some examples, the controller can compare the received information, or information derived from the received information after further processing, to one or more comparison values to determine an appropriate amount of one or more metabolic modulators to deliver to the user. The appropriate amount could be delivered immediately, at a next scheduled delivery time, or when a user next indicates they want a dose (e.g. by interacting with a user interface or puff sensor).

The one or more comparison values comprise at least one value selected from the group comprising a baseline value, a target value, a threshold value, an upper range value, and a lower range value.

In some examples, a comparison between the received information, or information derived from the received information after further processing, and a baseline value provides an indication of how elevated the user's metabolic state is in comparison to a resting metabolic state. For example, if it is established based on the received information that the user's metabolic state is close to a baseline metabolic state of the user then the controller can determine that the user's metabolic state is close the rest state (where the baseline is measured while the user is at rest and/or corresponds to a minimum values measured for the metabolic state). In such a case, the controller can determine that it is appropriate that the user's intake of metabolic stimulant should be increased and/or that the user's intake of metabolic suppressant should be decreased. Alternatively, if it is established from the received information that the user's metabolic state is significantly above a baseline metabolic state of the user, then the controller can determine that the user's metabolic state is in an elevated state. In such a case, the controller can determine that it is appropriate that the user's intake of metabolic stimulant should be decreased and/or that the user's intake of metabolic suppressant should be increased. In some examples, the amount of one or more metabolic modulators provided to the user can be regulated dependent on how much the controller determines the user's metabolic state differs from that of the baseline state.

In some examples, a comparison between the received information, or information derived from the received information after further processing, and a target value provides an indication of how close the user's metabolic state is to a target metabolic state. For example, if it is established based on the received information that the user's metabolic state is less than a target metabolic state of the user then the controller can determine that it is appropriate that the user's intake of metabolic stimulant should be increased and/or that the user's intake of metabolic suppressant should be decreased. Alternatively, if it is established from the received information that the user's metabolic state is greater than a target metabolic state of the user, then the controller can determine that it is appropriate that the user's intake of metabolic stimulant should be decreased and/or that the user's intake of metabolic suppressant should be increased. In some examples, the amount of one or more metabolic modulators provided to the user can be regulated dependent on how close the controller determines the user's metabolic state is to that of the baseline state.

In some examples, a comparison between the received information, or information derived from the received information after further processing, and a threshold value provides an indication of a particular metabolic state of the user. For example, if the comparison establishes that a threshold value is exceeded then the controller can determine an appropriate amount of one or more metabolic modulators to provide to the user, whereas, if the comparison establishes that a threshold value is not exceeded then the controller can determine an different appropriate amount of one or more metabolic modulators to provide to the user.

Different threshold values can indicate different metabolic states. Furthermore, it will be appreciated that threshold values can be configured in relation to baseline and/or target metabolic states. For example a threshold value could be set as a percentage increase in relation to a baseline value. In some examples, the controller may determine that the metabolic state of the user is above a threshold and prevent or restrict further dosing of one or more metabolic stimulants until the metabolic state has reduced beneath a threshold. Alternatively, the controller may determine that the metabolic state of the user is below a threshold and prevent or restrict further dosing of one or more metabolic suppressants until the metabolic state has increased above a threshold.

In some examples, the controller can perform comparisons with multiple threshold values with a particular metabolic state being indicated dependent on which of the threshold values are exceeded or not exceeded. The controller can control the delivery of at least one metabolic modulator differently dependent on which thresholds are exceeded or not exceeded.

In some examples, a comparison between the received information, or information derived from the received information after further processing, and an upper range value and/or a lower range value provides an indication of a particular metabolic state of the user. For example, if the comparison establishes that the metabolic state of the user is between the range values, the controller can determine an appropriate amount of one or more metabolic modulators to provide to the user. In some examples, a value falling within a range can indicate that the metabolic state of the user is close to a desired state. In some examples, a value greater than an upper range value can indicate that the metabolic state of the user is elevated in comparison to a desired state. In some examples, a value greater than an lower range value can indicate that the metabolic state of the user is lower than a desired state. Furthermore, it will be appreciated that range values can be configured in relation to baseline and/or target metabolic states. For example, upper and lower range values could be set as percentage increases in relation to a baseline value. For example, upper and lower range values could be set as percentage changes in relation to a target value.

In some examples the controller is configured to determine an amount (e.g. a dose) of one or more metabolic modulators to be provided or delivered to a user. In some examples the dose size is calculated by the controller based on the received information relating to the metabolic state of the user. In some examples, the dose is supplied in a single release. In some examples, a first dose is provided at a first time and, subsequently, a second dose is provided at a second time if data received by the sensor after the first time indicates that the metabolic state has not changed by a predicted or threshold amount. In some examples, the dose size is pre-determined (e.g. fixed). In these examples, the controller may provide a number of doses of the pre-determined size to provide a determined amount of one or more metabolic modulators.

In some examples, the delivery device can comprise two or more sources or substances, each containing a different amount of metabolic modulator(s). In some examples, the two or more sources can be in a liquid form and each may be provided in respective reservoirs. In some examples, the two or more sources may be in a solid or semi-solid form (e.g. a gel) and each may be provided in a chamber or wrapper. In some examples, the two or more sources may include a first source in a first state (e.g. a liquid state) and a second source in a second state (e.g. a gel state).

In some examples, each of the two or more sources is associated with a separate delivery mechanism, such as an aerosol generator. Alternatively, the same delivery mechanism can be used with two or more sources as long as there is a mechanism(s) for selectively controlling delivery from each source. For example, where the two sources are to be aerosolised and the sources are liquid, a (or multiple) release mechanism(s) may release liquid from one or both of the sources into a region adjacent an aerosol generator. The controller can control the delivery of at least one metabolic modulator by controlling delivery from the two or more sources.

In some examples a first substance comprises at least one metabolic modulator, whilst a second substance does not comprise a metabolic modulator or comprises a reduced amount of the at least one metabolic modulator. In these examples, the controller 120 can control the amount of the at least one metabolic modulator delivered to the user by controlling the delivery from each of the first and second substances. Furthermore, delivering a substance containing a reduced amount or no metabolic modulator in addition to the substance containing a larger amount of metabolic modulator can ensure that a user experiences substantially no difference in the total amount of substance being delivered. This can ensure a more consistent user experience.

In some examples, a first substance can contain at least one metabolic stimulant and a second substance can contain at least one metabolic suppressant. The controller can vary the amount of the first and second substance delivered to the user to promote a desired change in the metabolic rate of the user. For example more metabolic stimulant that metabolic suppressant can be provided if it is desired to increase the metabolic rate of the user, or alternatively, more metabolic suppressant can be provided if it is desired to decrease the metabolic rate of the user. In some examples where the delivery device is an aerosol provision device, the controller can control the amount of aerosol produced from a first reservoir and the amount of aerosol produced from the second reservoir to promote a change in the metabolic state of a user (e.g. by providing a larger ratio of aerosol from the first reservoir in comparison to aerosol from the second reservoir to promote an increase in the metabolic rate of the user, and by providing a smaller ratio of aerosol from the first reservoir in comparison to aerosol from the second reservoir to promote a decrease in the metabolic rate of the user).

FIG. 2 is an abstract graphical representation of the metabolic rate of a user over time. Three metabolic rate levels “A”, “B”, and “C” are shown in FIG. 2 as dashed lines. By metabolic rate level it is meant a line of constant metabolic rate (i.e. a horizontal line). Three example user metabolic rate profile “a”, “b”, and “c” are shown in FIG. 2 as solid lines. A user metabolic rate profile depicts an example change in metabolic rate of a user over time.

In other words, a user metabolic rate profile depicts the change in metabolic rate at various times for a hypothetical user. As discussed above a controller can be configured to provide an amount of one or more metabolic modulators (i.e. metabolic stimulants and metabolic suppressants) dependent on the user's metabolic profile as indicated by each of the example metabolic rate profiles. By provide, it is meant that the controller may either directly provide or dose a user with the altered amount of metabolic modulator(s) (e.g. where the delivery component is able to deliver metabolic modulator to a user without user interaction) or may indirectly provide or dose a use with the altered amount of metabolic modulator(s) (e.g. where the delivery component requires a user interaction to be able to deliver metabolic modulator to a user).

In accordance with the second example user metabolic rate profile “a”, the controller may identify that the user's metabolic rate is at a level “B” which may correspond to a lower threshold or a lower range value. In some examples, in response to the controller identifying, or being otherwise informed of (e.g. by a sensor), the metabolic rate of the user is at the level “B”, the controller is configured to increase the amount of metabolic stimulant and/or decrease the amount of metabolic suppressant provided to a user. In some examples, the controller is configured to continuously or repeatedly provide the increased amount of metabolic stimulant and/or the decreased amount of metabolic suppressant until the controller identifies, or is otherwise informed that, the metabolic rate of the user reaching or exceeding a level “C”. In some examples, the controller may provide a default amount metabolic modulator(s) once the controller identifies, or is otherwise informed of, the metabolic rate of the user exceeds the metabolic rate level “C”. The level “C” may correspond to an upper threshold or a upper range value.

In some examples, the controller is configured to provide the increased amount of metabolic stimulant and/or the decreased amount of metabolic suppressant for a period of time and/or for a number of uses or doses after the comptroller identifies that the user's metabolic rate is at a level “B”. In some examples, the controller may provide the increased amount of metabolic stimulant and/or the decreased amount for an additional period of time and/or for a further number of uses or doses if the comptroller identifies that the user's metabolic rate has not increased above a specified rate (e.g. a different threshold value between level “B” and “C”) after the provision of the increased amount of metabolic stimulant and/or the decreased amount of metabolic suppressant for the first period of time and/or for the first number of uses or doses. In other words, while the user's metabolic rate is below a first level, the controller can provide an increased amount of one or more metabolic stimulators to increase the user's metabolic rate, and/or can restrict the amount of one or more metabolic suppressants to prevent or limit any suppressing effect on the user's metabolic rate.

In accordance with the second example user metabolic rate profile “b”, the controller may identify that the user's metabolic rate is at a level “C” which may correspond to an upper threshold or an upper range value. In some examples, in response to the controller identifying, or being otherwise informed of (e.g. by a sensor), the metabolic rate of the user is at the level “C”, the controller is configured to decrease the amount of metabolic stimulant and/or increase the amount of metabolic suppressant provided to a user. In some examples, the controller is configured to continuously or repeatedly provide the decreased amount of metabolic stimulant and/or the increased amount of metabolic suppressant until the controller identifies, or is otherwise informed of, the metabolic rate of the user reaching or being lower than a level “B”. In some examples, the controller may provide a default amount metabolic modulator(s) once the controller identifies, or is otherwise informed that, the metabolic rate of the user exceeds the metabolic rate level “B”. The level “B” may correspond to a lower threshold or a lower range value.

In some examples, the controller is configured to provide the decreased amount of metabolic stimulant and/or the increased amount of metabolic suppressant for a period of time and/or for a number of uses or doses after the comptroller identifies that the user's metabolic rate is at a level “C”. In some examples, the controller may provide the decreased amount of metabolic stimulant and/or the increased amount for an additional period of time and/or for a further number of uses or doses if the comptroller identifies that the user's metabolic rate has not decreased below a specified rate (e.g. a different threshold value between level “B” and “C”) after the provision of the decreased amount of metabolic stimulant and/or the increased amount of metabolic suppressant for the first period of time and/or for the first number of uses or doses. In other words, while the user's metabolic rate is above a level, the controller can restrict the amount of one or more metabolic stimulators to prevent or limit any stimulating effect on the user's metabolic rate, and/or can provide an increased amount of one or more metabolic suppressants to decrease the user's metabolic rate.

In accordance with the first example user metabolic rate profile “c”, a user's metabolic rate may start at a first level before dropping over time towards a baseline level “A” (which may have been calibrated as described above). In some examples, in response to the controller identifying, or being otherwise informed of (e.g. by a sensor), the drop in metabolic rate towards the baseline value “A” (e.g. when it drops below the first level “B”) and/or the metabolic rate reaching the baseline value “A”, the controller can increase the amount of metabolic stimulant and/or decrease the amount of metabolic suppressant provided to a user. In some examples, the controller is configured to continuously or repeatedly provide the increased amount of metabolic stimulant and/or the decreased amount of metabolic suppressant until the controller identifies, or is otherwise informed of, the metabolic rate of the user reaching or exceeding a metabolic rate level “B”. In some examples, the controller is configured to provide the increased amount of metabolic stimulant and/or the decreased amount of metabolic suppressant for a period of time and/or for a number of uses or doses after the comptroller identifies that the user's metabolic rate is at a level “A”. In some examples, the controller is configured to provide the increased amount of metabolic stimulant and/or the decreased amount for an additional period of time and/or for a further number of uses or doses if the comptroller identifies that the user's metabolic rate has not increased above a specified rate (e.g. a different threshold value between level “B” and “A”) after the provision of the increased amount of metabolic stimulant and/or the decreased amount of metabolic suppressant for the first period of time and/or for the first number of uses or doses.

In some examples, the controller is configured to provide a default amount metabolic modulator(s) once the controller identifies, or is otherwise informed that, the metabolic rate of the user exceeds the metabolic rate level “B”. In some examples, the controller is configured to provide an increased amount of metabolic stimulant and/or a decreased amount of metabolic suppressant while the metabolic rate of the user is above “B” but below “C” in accordance with the functionality described in relation to user metabolic rate profile “a”. In these examples, the increased amount of metabolic stimulant may be less than the amount of metabolic stimulant provided when the user's metabolic rate is less than “B” but greater than a default amount; and/or the decreased amount of metabolic suppressant may be greater than the amount of metabolic suppressant provided when the user's metabolic rate is less than “B” but less than a default amount. In other words, while the user's metabolic rate is below a first level, the controller can provide a first increased amount of one or more metabolic stimulators to increase the user's metabolic rate, and/or provide a first decreased amount of one or more metabolic suppressants to prevent or limit any suppressing effect on the user's metabolic rate; and while the user's metabolic rate is above a first level but below a second level, the controller can provide a second increased amount of one or more metabolic stimulators to increase the user's metabolic rate, and/or provide a second decreased amount of one or more metabolic suppressants to prevent or limit any suppressing effect on the user's metabolic rate, where the second increased amount is less than the first increased amount and the second decreased amount is greater than the first decreased amount.

### Delivery Systems

In accordance with example embodiments of the present disclosure, a delivery system comprises a controller configured to receive information or data relating to the metabolic state of the user from at least one sensor. The controller is configured to control delivery of at least one metabolic modulator to the user in response to, or based on, the information relating to the metabolic state of the user. A delivery system having a controller configured in this way is operable to respond to provide a level of control of the metabolic state of a user.

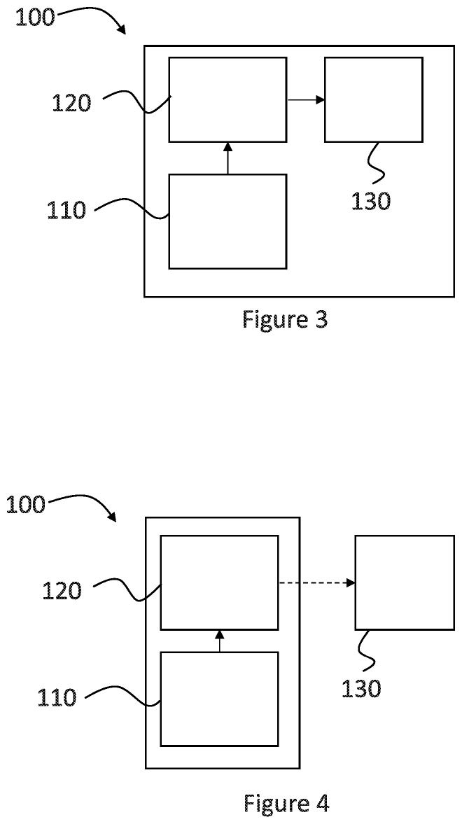

FIG. 3 is a schematic diagram of an exemplary delivery system in accordance with the present invention. The delivery system 100 of FIG. 3 comprises a sensor device or module 110, a controller 120, and a delivery component 130. In the example of FIG. 3 the sensor component 110, the controller 120 and the delivery component 130 are provided as part of a single device (e.g. they comprises components which are fixedly or removably connectable, and that are configured to be attached together in normal use). In some examples, one or more of the sensor component 110, the controller 120 and the delivery component 130 may be provided in a device housing or body, or otherwise may be attached to a device housing or body.

The sensor component 110 comprises at least one sensor. In some examples the sensor component 110 comprises a single sensor. In some examples, the sensor component 110 comprises two or more sensors. In some examples, the two or more sensors measure at least two different physical characteristics. In some examples, one or more additional sensors are provided in a second physically separate sensor component (e.g. two or more sensors may be provided at different locations within or on the surface of a housing or body of the deliver system). In some examples, the two or more sensors of the sensor component 110 are provided as part of a same component (for example they may be provided on a same PCB or chip).

In some examples, the at least one sensor of the sensor component 110 can output unprocessed or raw sensor readings corresponding to measurements obtained by the at least one sensor. In some examples, the at least one sensor of the sensor component 110 can output a processed sensor reading corresponding to measurements obtained by the at least one sensor. In these examples, the at least one sensor can be configured to have a level of processing capability to enable them to perform any necessary processing. In some examples, measurements can be sampled or averaged to provide the output sensor readings. In some examples, measurements can be converted (in some cases after sampling or averaging) to provide the sensor reading (e.g. voltage measurements measured by a sensor could be converted into a measurement of heart rate by the sensor). It will be appreciated that where there are two or more sensors, each sensor may operate differently such that a first sensor may output raw sensor readings whilst a second sensor may output

The controller 120 (e.g. a control unit, a processing unit, or a computing unit) is configured to receive sensor readings output by the sensor component 110 (e.g. from the at least one sensor of the sensor component 110). The controller 120 is configured to receive the sensor readings via either wired or wireless communication. It will be appreciated that for the system of FIG. 3, a wired connection (e.g. electrically connected paths formed by distinct wires or conductive pathways provided on a circuit board) is preferable as the wired connections can be provided substantially internally to the housing or body, such that they are user does not normally interact with them and without having to provide more mechanisms for facilitating wireless connections.

The controller 120 is configured to control delivery of at least one metabolic modulator to the user. In some examples, the sensor readings comprise information relating to the metabolic state of the user. In some examples, the controller 120 is configured to process the sensor readings received from the sensor component 110 to determine or establish information relating to the metabolic state of the user. For example, the controller 120 may compare sensor readings to a baseline and/or convert the sensor readings into a format indicative of metabolic state to determine or establish information relating to the metabolic state of the user.

The delivery component 130 is in wired or wireless communication with the controller 120. The delivery component 130 is operable to receive a command or signal from the controller 120, and to deliver at least one metabolic modulator in response to the command or signal. In some examples, the controller 120 can emit the command or signal to the delivery component 130 upon determining that the at least one metabolic modulator should be supplied to the user. In some other examples, the controller 120 can emit the command or signal to the delivery component 130 upon determining that a user has interacted with the delivery system (e.g. by interacting with a user interface and/or activating a sensor).

In some examples, such as those in accordance with FIG. 1, the source of the metabolic modulator is a liquid source. In other examples, the source of the metabolic modulator is a non-liquid source. For example, the source may be in a solid or semi-solid form, such as a gel. In some examples, the delivery component comprises the source of the metabolic modulator. For example the delivery system may comprise a cavity or chamber in which the source is contained. In some examples, the delivery component is configured to receive the source or otherwise connect to a consumable containing the source. In some examples, the source can be provided in a container or cartridge which is attached to the delivery system. In some examples, the delivery component comprises a chamber or cavity for receiving the source. In some examples the source is provided into a chamber or cavity of the delivery component by itself (e.g. a liquid may be fed into the chamber or cavity, or a solid material, such as a tobacco material, may be placed into the chamber or cavity), whilst in other examples the source may be provided, at least partially, in a wrapper, container or other barrier material which is inserted into the chamber or cavity of the delivery component (in some examples, the delivery component comprises a mechanism for breaking or bypassing a barrier material to allow the source to come into contact with a delivery mechanism such as a heater).

In some examples the source of metabolic modulator comprises multiple compounds which have a metabolic effect on a user. For example, the source can comprises two or more metabolic stimulants, two or more metabolic suppressants or a combination of one o more metabolic stimulants and one or more metabolic suppressants. It will be appreciated that the combination of two metabolic stimulants, or alternatively two or more metabolic suppressants, may have a greater effect on the metabolic rate than a single metabolic stimulant, or suppressant, respectively. It will also be appreciated that the combination of a weak metabolic suppressant with a strong metabolic stimulant, or vice versa, will lessen the effect of the metabolic modulator on the user.

In some examples, a consumable containing the source comprises a identification means and the delivery component is configured to identify the source based on the identification means. In some examples, the identification means comprises a RFID chip, and the delivery component is configured to read the RFID chip. In some examples, the identification means comprises a physical marker or colour, and the delivery component is configured to identify the physical marker or colour. In some examples, the identification means comprises a resistive component, and the delivery component is configured to form an electric circuit with the resistive component and to measure an electrical characteristic. In some examples, the identification means is interpreted by a user or controller 120, and the delivery component is provided with information relating to the identification means which is used by the delivery component to identify the source.

By identify the source, it is meant that the delivery component can identify a type of source, a composition of the source, one or more metabolic modulators contained within the source, an amount of one or more metabolic modulators contained within the source, a strength (i.e. concentration) of one or more metabolic modulators contained within the source, and/or an estimated effect of the source. In some examples, the delivery component will communicate information relating to the consumable, based on the identification means, to the controller 120.

In some examples, the delivery component comprises a delivery mechanism for delivering the metabolic modulator contained in the source to the user. In some examples, the delivery component is configured to activate a delivery mechanism for delivering the metabolic modulator contained in the source to the user. For example, the delivery component may be in electrical connection with a delivery mechanism for delivering the metabolic modulator contained in the source to the user. In some examples, the delivery mechanism is an aerosol generator, such as a heater or vibrating mesh. In some examples, the delivery mechanism is a substance release mechanism configured to dispense a substance in response to a command. Such a substance release mechanism may be for example a pump, syringe or similar.

In some examples, the command or signal results in the activation of the delivery mechanism for the period in which the signal is received. For example, the controller 120 can control a switch that provides power to the delivery component 130, with the delivery component 130 functioning to deliver at least one metabolic modulator as long as power is supplied (e.g. the delivery component could comprise a resistive heater which generates heat to vaporise a solution containing the at least one metabolic modulator when a current is supplied through the heater).

In some examples, the delivery component 130 receives the signal or command from the controller 120 and determines or establishes an appropriate amount of the at least one metabolic modulator to deliver. For example the delivery component 130 can process the signal or command to determine or estimate an amount of the at least one metabolic modulator that should be delivered. In contrast to the situation in the paragraph above, where the command or signal results in activation of the delivery mechanism for the period in which the signal is received, in these examples, the command or signal can be delivered over a period which is far shorter than the period in which the delivery mechanism of the delivery component 130 is active. For example, the command or signal could indicate that the delivery component 130 should delivery the at least one metabolic modulator; and in response the delivery component 130 could enact one of a number of delivery protocols or programs (e.g. the delivery component could active a delivery mechanism for a period of 30 seconds after receiving the command or signal).

In a first example, a delivery system 100 in accordance with FIG. 3 could be an aerosol provision system (e.g. similar to that described in relation to FIG. 1). Such a delivery system 100 could comprise the sensor component 110 and the controller 120 in the body portion 20, while the delivery component 130 could be provided by the cartomiser portion 130. An example sensor component 110 could comprise one or more sensors selected from the group comprising a temperature sensor (e.g. for measuring a temperature of the user when the user holds the device in their hand), a heart rate sensor (e.g. for measuring the heart rate of the user when the user holds the device in their hand), an motion sensor (e.g. for measuring movement of the user). Where the sensor is a temperature or heart rate sensor (or other sensor requiring proximity to the user) this could be provided on a surface portion of the body portion 20, which is expected to be covered by the user's hand when the device is gripped by the user in use. Where the sensor is a motion sensor (or other sensor not requiring proximity to the user), the sensor can be provided internally to the body such that it is better protected from damage.

For a delivery system 100 in accordance with the first example, the sensor component 110 can measure a physical characteristic and output sensor reading to the controller 120. The controller 120 can read and/or analyse the sensor readings to establish, determine or estimate information relating to a metabolic state of the user. The controller 120 can determine an appropriate command or signal with which to control the delivery of the at least one metabolic modulator based on the information. The controller 120 can control the delivery of the at least one metabolic modulator by controlling the activation of an aerosol generator of the cartomiser 30 when a user next uses the system (e.g. by drawing on the device to activate a pressure sensor and/or interacting with a user interface).

In some examples, the controller 120 can select a power, from a plurality of (continuous or discontinuous) power levels, to supply to the aerosol generator which can change the amount of aerosol produced. In these examples, if the controller 120 determines a larger amount of the one or more metabolic modulator should be delivered, the controller 120 increases the power; and if the controller 120 determines a smaller amount of the one or more metabolic modulator should be delivered, the controller 120 decreases the power.

In some examples, the controller 120 can alter a time period in which power is supplied to the aerosol generator (e.g. the time period can be a maximum time period in which power is supplied, or the time period can be a time period in which power is supplied at a first level, and any power supplied after the time period is supplied at a second, lower, level). In these examples, if the controller 120 determines a larger amount of the one or more metabolic modulator should be delivered, the controller 120 increases the time period;

and if the controller 120 determines a smaller amount of the at least one metabolic modulator should be delivered, the controller 120 decreases the time period.

In a second example, a delivery system 100 in accordance with FIG. 3 could be an aerosol-free delivery system such as a patch or implant. A delivery system 100 in accordance with the second example, can comprise the sensor component 110, the controller 120, and delivery component 130 in a single body. Where the delivery system 100 is a patch, this can be provided on the skin of the user such that the delivery component 130 can transdermally deliver the at least one metabolic modulator. An example sensor component 110 could comprise one or more sensors selected from the group comprising a temperature sensor (e.g. for measuring a temperature of the user), a heart rate sensor (e.g. for measuring a heart rate of the user), a motion sensor (e.g. for measuring movement of the user).

For a delivery system 100 in accordance with the second example, the sensor component 110 can measure a physical characteristic and output sensor reading to the controller 120. The controller 120 can read and/or analyse the sensor readings to establish, determine or estimate information relating to a metabolic state of the user. The controller 120 can control the delivery of the at least one metabolic modulator based on the information (e.g. by issuing an appropriate command or signal to the controller 120). The controller 120 can control the delivery of the at least one metabolic modulator by controlling the activation of a delivery mechanism of the delivery component 130. For example, the delivery component 130 can comprise a discharge mechanism for discharging a substance containing the at least one metabolic modulator into a portion of the patch adjacent to the skin of the user such that it can be transdermally provided to the user.

FIG. 4 is a schematic diagram of an exemplary delivery system in accordance with the present invention. The delivery system 100 of FIG. 4 comprises a sensor component 110, a controller 120, and a delivery component 130. In the example of FIG. 4 the sensor component 110 and the controller 120 are provided as part of a single device, whilst the delivery component 130 is, or is provided as part of, a separate device (such a delivery component can be termed a delivery device). The controller 120 is configured to communicate with the delivery component 130 via a wired or wireless connection.

Preferably, the controller 120 is configured to communicate via a wireless connection to avoid wires or similar connecting the controller 120 to the delivery component 130 which can be inconvenient for the user.

By separating the delivery component 130 from the controller 120 and the sensor component 110, the device containing the one or more sensors is reduced in size. A smaller device can be attached to the user (e.g. as a “wearable” device) as the reduced size means that the device has a lesser impact on the user's normal activities. If the device containing the one or more sensors is attached to the user then measurements can be taken more reliably. For example, the sensors are typically attached in a single position for a longer period of time which aids determining a baseline and/or changes to the metabolic state of the user.

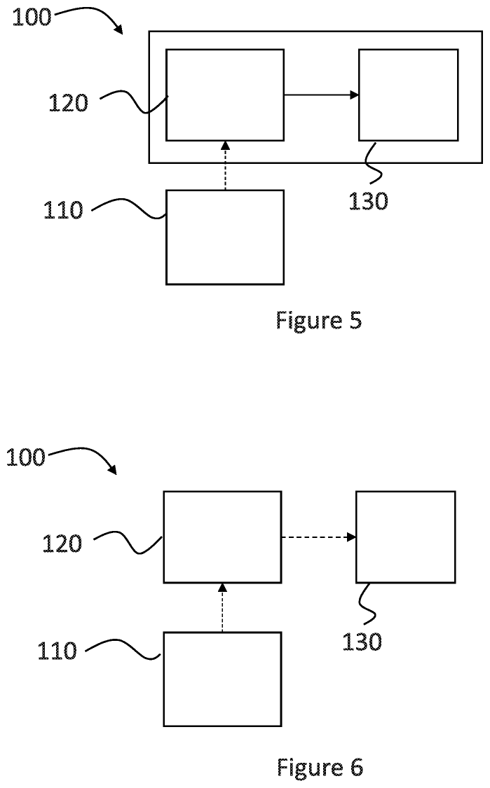

FIG. 5 is a schematic diagram of an exemplary delivery system in accordance with the present invention. The delivery system 100 of FIG. 5 comprises a sensor component 110, a controller 120, and a delivery component 130. In the example of FIG. 5 the delivery component 130 and the controller 120 are provided as part of a single device, whilst the sensor component 110 is, or is provided as part of, a separate device. The controller 120 is configured to communicate with the sensor component 110 via a wired or wireless connection. Preferably, the controller 120 is configured to communicate via a wireless connection to avoid wires or similar connecting the controller 120 to the sensor component 110 which can be inconvenient for the user.

By separating the sensor component 110 from the controller 120 and the delivery component 130, the device containing the one or more sensors is further reduced in size. A smaller device can be attached to the user (e.g. as a “wearable” device) as the reduced size means that the device has a lesser impact on the user's normal activities. If the device containing the one or more sensors is attached to the user then measurements can be taken more reliably. For example, the sensors are typically attached in a single position for a longer period of time which aids determining a baseline and/or changes to the metabolic state of the user.

FIG. 6 is a schematic diagram of an exemplary delivery system in accordance with the present invention. The delivery system 100 of FIG. 6 comprises a sensor component 110, a controller 120, and a delivery component 130. In the example of FIG. 6 the sensor component 110, the controller 120 and the delivery component 130 are each provided by separate devices. The controller 120 is configured to communicate with the sensor component 110 and the delivery component 130 via a wired or wireless connection.

Preferably, the controller 120 is configured to communicate via a wireless connection to avoid wires or similar connecting the controller 120 to the sensor component 110 and the delivery component 130 which can be inconvenient for the user.

By separating the sensor component 110 from the controller 120 and the delivery component 130, the device containing the one or more sensors is further reduced in size. A smaller device can be attached to the user (e.g. as a “wearable” device) as the reduced size means that the device has a lesser impact on the user's normal activities. If the device containing the one or more sensors is attached to the user then measurements can be taken more reliably. For example, the sensors are typically attached in a single position for a longer period of time which aids determining a baseline and/or changes to the metabolic state of the user. Furthermore, by separating the controller 120 from the delivery component 130, a conventional computing device, such as a smartphone or similar, can be programmed to provide the functionality required of the controller 120.

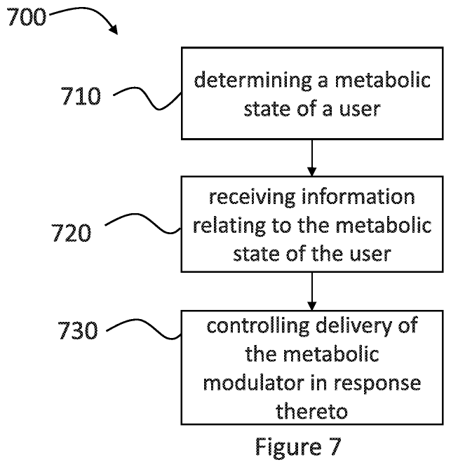

FIG. 7 schematically represents a method 700 of controlling an aspect of the delivery system in accordance with certain embodiments of the disclosure. The delivery system may be in accordance with the any of delivery systems of FIGS. 3 to 6, and as such comprise a sensor component 110 configured to determine a metabolic state of a user, a delivery component 130 configured to deliver a metabolic modulator and a controller 120 configured to receive information relating to the metabolic state of the user and to control delivery of the metabolic modulator in response thereto.

The method 700 starts at step 710 with determining a metabolic state of the user. By determine a metabolic state of the user it is meant that the sensor component 110 measures a parameter or characteristic associated directly or indirectly with the metabolic rate of the user. In some examples, the sensor component 110 may perform analysis or processing on the measured parameter or characteristic whilst in other examples, the sensor component 110 may store and/or transmit the measured parameter or characteristic in a raw format.

The method 700 continues at step 720 with receiving information relating to the metabolic state of the user. By receiving information relating to the metabolic state of the user it is meant that the controller 120 receives the measurement of the parameter or characteristic associated directly or indirectly with the metabolic rate of the user from the sensor component 110. The measurement may be in a processed or unprocessed format. For examples the transmitted measurement may be an average of a plurality of measurements.

The method 700 continues at step 730 with controlling delivery of the metabolic modulator in response thereto. By controlling delivery of the metabolic modulator in response thereto it is meant that the controller 120 analyses the information received at step 720 to determine an appropriate level or amount of metabolic modulator to deliver, and instructs or commands the delivery component 130 to delivery the appropriate level or amount of metabolic modulator. In some examples, the delivery component 130 will deliver an amount of metabolic modulator substantially immediately in response to receiving the instruction or command. In some examples, the delivery component 130 will deliver an amount of metabolic modulator corresponding to the instruction or command in response to a user interaction with the delivery component 130.

Thus there has been described a delivery system for a metabolic modulator comprising: a sensor component 110 configured to determine a metabolic state of a user; a delivery component 130 configured to deliver a metabolic modulator; and a controller 120 configured to receive information relating to the metabolic state of the user and to control delivery of the metabolic modulator in response thereto.

Thus there has been described a controller 120 for a delivery system for a metabolic modulator, the controller 120 configured to: receive information relating to the metabolic state of the user from a sensor component 110 configured to determine a metabolic state of a user; and control delivery of the metabolic modulator via in response thereto.

Thus there has been described a delivery component 130 for a delivery system for a metabolic modulator, the delivery component 130 configured to deliver a metabolic modulator in response to information relating to the metabolic state of the user.

Thus there has been described a method of operating a delivery system for a metabolic modulator, the delivery system comprising a sensor component 110 configured to determine a metabolic state of a user, a delivery component 130 configured to deliver a metabolic modulator and a controller 120 configured to receive information relating to the metabolic state of the user and to control delivery of the metabolic modulator in response thereto, the method comprising: determining a metabolic state of a user; and receiving information relating to the metabolic state of the user; and controlling delivery of the metabolic modulator in response thereto.

Thus there has also been described means for delivering a metabolic modulator comprising: sensor means configured to determine a metabolic state of a user; delivery means configured to deliver a metabolic modulator; and control means configured to receive information relating to the metabolic state of the user and to control delivery of the metabolic modulator in response thereto.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. A delivery system for a metabolic modulator comprising:
a sensor component configured to determine a metabolic state of a user;
a delivery component configured to deliver a metabolic modulator; and
a controller configured to receive information relating to the metabolic state of the user and to control delivery of the metabolic modulator in response thereto.

2. The delivery system of claim 1, wherein the controller is configured to use the received information relating to the metabolic state of the user to perform a comparison to one or more comparison values, and to control delivery of the metabolic modulator based on the comparison.

3. The delivery system of claim 2, wherein the one or more comparison values comprise at least one value selected from the group comprising a baseline value, a target value, a threshold value, an upper range value, and a lower range value.

4. The delivery system of claim 2, wherein the controller is configured to establish a metabolic rate of the user based on the received information relating to the metabolic state of the user, wherein the comparison comprises comparing the established metabolic rate to the one or more comparison values.

5. The delivery system of claim 1, wherein the controller is configured to control delivery of the metabolic modulator by determining a delivery parameter for delivering the metabolic modulator, and wherein the delivery component is configured to deliver the metabolic modulator based on the determined delivery parameter.

6. The delivery system of claim 5, wherein the delivery component is configured to deliver a first amount of metabolic modulator when the determined delivery parameter is a first delivery parameter and to deliver a second amount of metabolic modulator when the determined delivery parameter is a second delivery parameter, the first amount greater than the second amount.

7. The delivery system of claim 1, wherein the delivery component is configured to deliver a replacement substance at least in part in place of the metabolic modulator, and wherein the controller is configured to control delivery of the replacement substance in response to the information relating to the metabolic state of the user.

8. The delivery system of claim 7, wherein the replacement substance is a second metabolic modulator.

9. The delivery system of claim 8, wherein the second metabolic modulator is configured to cause a smaller change in the metabolic state of the user than the first metabolic modulator.

10. The delivery system of claim 8, wherein the metabolic modulator comprises a metabolic stimulant and the second metabolic modulator comprises a metabolic suppressant.

11. The delivery system of claim 1, wherein the delivery component comprises a reservoir for the metabolic modulator.

12. The delivery system of claim 1, wherein the delivery component is physically separate from the sensor component.

13. The delivery system of claim 1, wherein the controller is physically separate from the sensor component and/or the delivery component.

14. The delivery system of claim 1, wherein the sensor component comprises one or more sensors selected from the group comprising a vibration sensor, an optical sensor, a temperature sensor, a motion sensor, a pressure sensor, and a chemical sensor.

15. The delivery system of claim 1, wherein the delivery component is configured to deliver the metabolic modulator during a usage event based on an interaction with the user.

16. A method of operating a delivery system for a metabolic modulator, the delivery system comprising a sensor component configured to determine a metabolic state of a user, a delivery component configured to deliver a metabolic modulator and a controller configured to receive information relating to the metabolic state of the user and to control delivery of the metabolic modulator in response thereto, the method comprising:
determining a metabolic state of a user; and
receiving information relating to the metabolic state of the user; and
controlling delivery of the metabolic modulator in response thereto.

17. A controller for a delivery system for a metabolic modulator, the controller configured to:
receive information relating to the metabolic state of the user from a sensor component configured to determine a metabolic state of a user; and
control delivery of the metabolic modulator via in response thereto.

18. A delivery component for a delivery system for a metabolic modulator, the delivery component configured to deliver a metabolic modulator in response to information relating to the metabolic state of the user.

19. (canceled)
